(19) United States
(12) Patent Application Publication
(10) Pub. No.: US 2009/0029448 A1
(43) Pub. Date: Jan. 29, 2009

(54) Compost Heat Recovery System and Method

(75) Inventor: Conrad Allain, Riverview (CA)

Correspondence Address: THE WEBB LAW FIRM, P.C., 700 KOPPERS BUILDING, 436 SEVENTH AVENUE, PITTSBURGH, PA, 15219, US

(73) Assignee: GREATER MONCTON SEWERAGE COMMISSION, Riverview (CA)

(21) Appl. No.: 12/164,607

(22) Filed: Jun. 30, 2008

(30) Foreign Application Priority Data
Jul. 27, 2007 (CA) 2593270

(51) Int. Cl. B09B 3/00; C12M 1/00; F28D 15/00

(52) U.S. Cl. 435/262.5; 435/290.1; 165/104.19

(57) ABSTRACT

In a composting system for outdoor operation for large-scale composting of municipal and other wastes, one or more large compost piles or windrows are supported on a pad, and covered with a removable cover. Fluid circulation pipes are embedded within the pad, to recover heat generated by aerobic decomposition of the organic matter and to transfer the recovered heat to a region of the pad underlying an edge region of the cover. The recovered heat warms the edge region, to thaw snow and ice which may have built up on the cover at the edge region, permitting the user to easily remove the cover without damage. A computerized controller operates the system, including circulation of fluid from selected central zones to selected edge zones, with real time data collection of temperature at critical locations and optional serial extraction and application of heat from and to multiple regions on the pad.

### CROSS-REFERENCE TO RELATED APPLICATION

This application claims the benefit under the Paris Convention of Canadian patent application no 2,593,270, filed on Jul. 27, 2007. The contents of said application are incorporated herein by reference.

### FIELD OF THE INVENTION

The invention relates to large-scale composting systems, for composting of biosolids and other wastes, in particular systems intended to be used outdoors on a solid slab in a cold climate. The invention further relates to methods and processes for large-scale composting of municipal and other wastes.

### BACKGROUND OF THE INVENTION

Modern municipal waste management includes as an integral component the composting of biological wastes. Such wastes include biosolids generated as a byproduct of waste water treatment. Compostable biosolids include the nutrient-rich organic matter generated by waste water treatment (‘green waste’), which constitutes a valuable resource for fertilizer and soil improvement. Other compostable wastes can also be generated or isolated from the waste stream. As used herein, the term “biosolid(s)” refers to any compostable matter. It is desirable to provide a composting step for treating biosolids, in order to reduce the level of pathogens in the material and to otherwise stabilize the substance, thus improving storage and handling while increasing the range of uses. In addition to wastewater-generated biosolids, large-scale composting is increasingly directed towards other municipal wastes. In addition, large-scale composting is carried out in the course of farming operations and various industrial, agricultural or forestry processes. The resulting compost has valuable uses in horticulture as mulch and in the manufacture of topsoil as well as other uses.

Modern composting techniques can result in the generation of substantial quantities of heat, in particular if the biosolids are mixed with an appropriate filler such as wood chips, bark and the like, which provide an open, porous structure to the pile. The composting process can be further driven by artificial aeration, such as by forcing air through the pile so as to encourage aerobic decomposition. By this means, extremely high temperatures which can reach or even exceed 75° C. may be achieved during at least some stages of the composting process. Such high temperatures are important for eliminating pathogenic micro-organisms. The heat generated during decomposition may be recovered and employed for a variety of uses.

Various systems have been proposed for capturing at least some of the heat generated during composting, and utilizing this recovered heat for various purposes. For example, in U.S. patent publication US 2003/0024686 A1 (Ouellette), a large-scale indoor composting system is disclosed, in which heat generated by a compost pile is captured in a first fluid medium flowing through an array of heat pipes, and transmitted to a secondary fluid medium where it is conveyed to a fluid storage tank. The captured heat is then used to supplement the existing water heating system of the structure, or is transmitted to an associated greenhouse or other enclosed space for space heating. Composting is carried out within a trench or on a pad.

U.S. Pat. No. 5,707,416 (Sudrabin) discloses a method and system for recovering heat generated during composting, in which the compost pile is placed on a perforated bed, with air being drawn downwardly through the pile and into the perforations, such that the air is warmed during its passage through the composting pile. The heated air may then be used in various applications, including being blown through a neighbouring compost pile for increasing the viability of the composting micro-organisms.

U.S. Pat. No. 6,399,359 (Hofstede) discloses a composting system in which the compost pile is covered with a weather-proof cover, sealed at its edges, for example by sand bags or other weights. Air is drawn through the compost pile, with the heated air then being drawn away via an air recycle pipe for space heating or other similar use.

PCT Application WO2007/006070 (Morrison) discloses a composting apparatus which has a container for containing a composting mass. A heat transfer device in the form of a coil or helical rib is provided for transferring heat from the hottest part of the composting mass throughout the composting mass to facilitate aerobic decomposition of the composting mass.

Prior art heat recovery systems have not been particularly well suited for the uses described above, and in particular if it is desired to provide a fully or partly automated operation of the system.

An efficient system for large-scale composting involves piling biosolids in long windrows, on a flat surface such as concrete pad. The windrows are then covered with a large air permeable tarp or membrane, which permits an outdoor composting operation which is operable even in cold climates. An example of a membrane that is well-suited for this purpose is a proprietary Gore™ cover system. This type of tarp-covered system has been applied successfully in several countries, primarily for green waste. It is preferable to operate this type of system outdoors, as it eliminates the need for large buildings and the extensive ventilation systems required in a closed environment. However, outdoor operation presents several challenges, in particular in cold climates. In particular, starting the composting process with a fresh pile can require an initial input of heat, in order to activate the composting micro-organisms.

A further challenge involves the build-up of snow and ice on the cover, in particular at the perimeter of the cover which rests directly on the concrete pad. This portion of the cover typically extends beyond the perimeter of the pile, and is anchored on the concrete pad with weights. However, snow and ice build-up on the peripheral portion can present a challenge to operators when it becomes necessary to remove the cover, for example in order to turn the pile or remove the composted matter. The built-up snow and ice has to be broken off and manually removed without damaging the cover material, involving significant manpower, as well as the potential for damaging the costly cover.

### SUMMARY

In order to overcome certain drawbacks and difficulties within the prior art, the present invention has as an object the provision of an improved composting system and method which is particularly suited for operation in cold climates. The improvements that are useful for cold climate composting include the use of pre-heated air for the aeration of the windrows and a compost heat transfer and storage system to pre-heat surfaces, and to melt snow and ice built-up. It is a further object to provide a system suitable for cold weather operation, in which the composting pile is covered with the tarp or membrane, for example, the Gore™ proprietary cover system. The heat generated during the composting operation may be selectively employed to melt ice and snow from the peripheral regions of the tarp or membrane which may have accumulated, so as to permit the operators to more easily remove the tarp for turning or removal of compost.

According to one general aspect, the invention relates to a composting system consisting of a solid pad or slab for supporting at least one pile of biosolids, preferably arranged as a long windrow. Optionally, the system includes a removable cover for selectively covering the pile, preferably consisting of a semi-permeable membrane specifically designed for outdoor composting operations. The cover when in use is positioned to fully cover a typical compost pile and to also extend past the edges of the pile such that a margin of the cover lies directly upon the pad adjacent to the base of the pile. This margin may be held down with weights or the like.

One or more arrays of pipes is embedded within the pad, preferably under the surface, with each array correspond with the location of a row region of the pad. Alternatively, the pipes may be installed so as to be in contact or near contact with the pad. The pipe array or arrays define one or more row regions on the pad surface for positioning of the compost piles thereon. The row regions are configured to support a desired configuration of compost pile. Most commonly, this will consist of an elongate windrow, which includes a central zone extending lengthwise along the pile, which underlies the main body of the compost piles, and edge zones forming the margins along either elongate side of the central zone. The edge zones extend beyond the expected edges of the compost piles so as to underlie a portion of the cover resting directly upon the pad. A heat-retaining fluid such as a glycol solution is provided from a source, and the fluid is circulated through the pipe array for transferring heat between different zones of the pad so as to selectively recapture heat from an actively composting pile and release it at a different zone of the pad. The recaptured heat recapture may be used to warm the edge zone of the pad so as to thaw the overlying cover where ice may have built up upon the cover margin, or a different row region to initiate the composting process.

The pipe array comprises at least one first sub-array of pipes located within the central zone of each row region, and one or more sub-arrays of pipes located within one or more of the edge zones. Each of said sub-arrays is independently connected to said source such that the fluid may be selectively circulated through the sub-arrays. Preferably, the pad includes multiple row regions, with multiple sub-arrays of pipes within the pad corresponding with the multiple central and edge zones. As such, the system is useful for handling multiple windrows of composting biosolids, with the heat generated by any particular windrow being capable of being directed towards an edge zone of any region of the pad, or stored for future use.

The pipes are part of a fluid circulation system that also includes one or more pumps or pump means are provided to pump the fluid through the pipe array, as well as valves to control the fluid flow so as to selectively circulate fluid through the arrays and sub-arrays of pipes.

The system further includes at least one temperature sensor which may be located within the fluid circulation of the pad, preferably at each central zone and edge zone to provide real time temperature data and a controller for controlling operation of the system in response to the input from the sensors as well as user input. Optionally, other sensors are provided, including additional temperature sensors, oxygen sensors within the compost pile and pressure sensors or flow rate sensors within the piping or elsewhere in the fluid circulation system.

A controller is provided which receives signals from the sensors and controls operation of the pump and valve means so as to selectively control the circulation of fluid from the central zones to the edge zones of the same or different row region, or between central zones of different row regions, so as to transfer heat generated by aerobic decomposition of compost at the central zone, to one or more edge zone in need of such generated heat, for example to thaw a portion of the cover for removal, or to the central zone of a row region remote from the source of the heat to initiate composting within a fresh pile. Typically, the controller consists of a PC specifically programmed to perform the operations described herein.

Preferably, the fluid source comprises an insulated liquid storage tank which includes temperature sensors for detecting the temperature of the liquid therein and for temporarily storing heated liquid for subsequent use when required. The pipe array consists of multiple pipes in fluid communication with the tank for incoming and outgoing fluid, so as to deliver or withdraw hot or cold liquid as required by the system. The pipes join with the tank at different elevations for taking advantage of vertical stratification of fluid within said tank based on differential temperatures of fluid therein.

The controller may include a visual display for providing a graphic illustration of the conditions of the system at the row regions of the pad, including the central and edge zones thereof, including the detected temperatures of the zones, the on/off status of the pump and valve means, and the enabled/disabled status of the central and edge zones. An enabled zone represents an active covered compost pile, and a disabled zone is indicative of an absence of an actively composting pile. The temperature, on/off status and enablement status is presented in real time, and permits user control over said system in response to said real time information. User control permits the ability to select the information to be displayed relating to the system in its entirety, or a portion of said system, or an individual compost pile region.

The control system may selectively control operation of the system according to one or more of a timed mode, a volume mode or a temperature mode. The selected mode or modes may be used to control circulation of the fluid within either of the centre or edge zones, or both, in any desired combination. The timed mode permits circulation of fluid within one or more zones for a selected time. The volume mode permits circulation of a selected volume of fluid within one or more zones in sequence, and the temperature mode permits circulation of fluid within one or more zones in sequence for so long as the detected temperature exceeds a selected minimum.

If the system includes a plurality of row regions, the fluid may be circulated serially within each selected region and zone in turn, optionally followed by a selected recovery period.

According to a general aspect, the invention relates to a system for composting of biosolids, comprising:

a) a pad for supporting at least one pile of said biosolids, said pad defining at least one row region on the surface thereof for receiving a corresponding pile, each row region comprising a central zone and one or more edge zones adjacent thereto,

b) a source of heat-retaining fluid suitable for transferring heat between said zones;

c) an array of pipes within said pad, in fluid communication with said source, said array comprising a first sub-array located within said central zone, and a second sub-array located within said edge zone, each sub-array being independently connected to said source, and valve means to selectively control the flow of fluid through the array;

d) pump means for moving said fluid through said pipes and said source;

e) at least one temperature sensor within each of said central zone or zones and said edge zone or zones, for transmitting signals in real time indicative of the temperature said pad, said fluid or said biosolids pile;

f) a controller for controlling operation of said system, said controller including means for selectively controlling the circulation of said fluid from said central zone to said edge zone so as to transfer heat generated by aerobic decomposition of said compost within said central zone, to an other region of said pad in need of such generated heat; and

g) signal transmission means between said controller and said temperature sensor, said pump means and said valve means.

According to another aspect, the invention relates to a method for composting of biosolids, comprising supporting at least one pile of biosolids on a pad as described above, including an array of pipes embedded in the pad with temperature sensors incorporated in the system as described above; optionally covering the pile with a removable membrane; supplying a heat-storing fluid which is suitable for transferring heat from one region of the pad to another; and controlling circulation of the fluid by receiving signal information from the temperature sensors and selectively controlling the circulation of fluid in response to the signal information such that fluid is selectively transferred from at least one, and preferably several central zones, to another zone of the pad in need of heat. The other zone which is the recipient of heat may consist of one or more edge zones. According to this aspect, the method permits heat generated by aerobic decomposition of biosolids within the central zone to be transferred to an edge zone in need of such generated heat, for example to thaw a portion of the cover which overlies the edge zone. According to another aspect, heat from one central zone may be transferred to another central zone so as to activate the composting process in the other zone.

The method also includes the steps of permitting user control of fluid circulation within the system. The controller may include a visual display associated as described above, with the method comprising operating the system with the aid of the visual display. The controller also includes the control and display functions described above. The method permits operation of the system according to one or more of a timed mode, a volume mode or a temperature mode, as described above.

Preferably, a plurality of said row regions is provided and the selected mode selectively circulates said fluid according to said selected mode serially within each selected region and zone in turn, optionally followed by a selected recovery period.

The present invention will now be further described and illustrate by way of a detailed description. It will be understood that this description is not intended to limit the scope of the invention, and is presented merely by way of illustration. Persons skilled in the art will understand that the elements described herein, including various means for performing functions and operations described herein, may be performed in a similar fashion by a variety of means known to those skilled in the art. Certain dimensions described herein are merely for illustration and are not intended to be limiting, since the present system may be readily scaled up or down as the need arises.

### BRIEF DESCRIPTION OF THE DRAWINGS

FIG. 1 is a layout view of a concrete pad with compost rows demarcated according to the invention.

FIG. 1a is an enlarged view of a portion of FIG. 1.

FIG. 2 is a sectional view of one windrow showing a portion of the concrete pad with the position of the aeration trenches, the embedded pipes and overall dimensions of the Central Zone and Edge Zones; a typical compost pile supported on the pad is also shown.

FIG. 3 is a side elevation view of a portion of the system along line 2-2 of FIG. 1, showing the concrete pad with four windrows thereon.

FIG. 4 is a schematic view of the fluid piping and storage components of the invention, related to one side or four compost windrows.

FIG. 5 is a schematic view of the fluid piping and connections in the trench and the arrangement of manifolds and supply/return headers.

FIG. 6 is a is a schematic view of an individual pipe and associated manifold connections according to the invention.

FIG. 7 is a side elevational view, in section, of a portion of the system showing the blowers and piping connections in relation to a covered windrow in place on the concrete pad.

FIG. 8 is a first screen shot of a video display-screen associated with the control system showing an overall view of all zones for the eight piles.

FIG. 9 is a second screen shot thereof; showing an overview of four piles and its associated pumping and storage system.

FIG. 10 is a third screen shot thereof; showing status of each zone under a windrow.

FIG. 11 is a fourth screen shot thereof.

FIG. 12 is a fifth screen thereof.

FIG. 13 illustrates operation of a portion of the control system 100.

FIG. 14 illustrates operation of a portion of the control system 100.

FIG. 15 illustrates operation of a portion of the control system 100.

FIG. 16 illustrates operation of a portion of the control system 100.

FIG. 17 illustrates operation of a portion of the control system 100.

### DETAILED DESCRIPTION

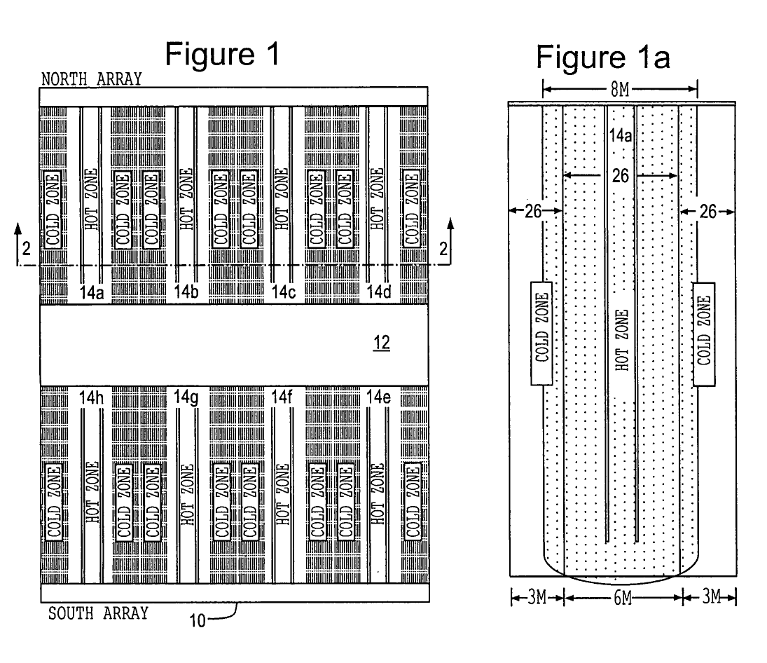

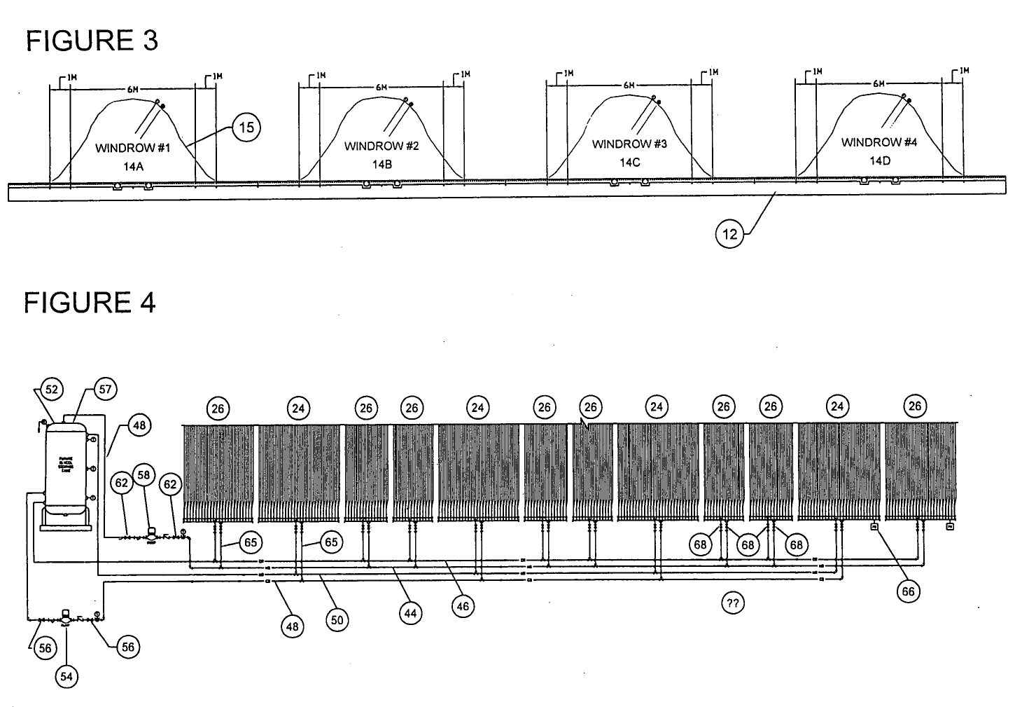

Turning to FIGS. 1, 1a, 2 and 3, a composting system 10 according to the invention comprises a rigid pad, such as a concrete slab 12, which supports a plurality of compost piles 14 arranged in elongate windrows 14(a) through 14(h). Each windrow is normally covered with a tarp 15, such as a proprietary Gore™ cover consisting of a semi-permeable membrane. The tarp retains heat and contains odours, while permitting release of excess moisture.

FIG. 1a shows in stippling the approximate location of a compost pile on the slab 12 which permits the system to be operated as described herein. While it will be understood that such piles will vary in size, the embodiment described herein operates particularly well when a pile having the approximate dimensions described herein is provided. In the non-limiting example described herein, each windrow when seen in cross section has a central region which includes the majority of the mass of the pile, and which is where most or all of the heat-generating composting activity occurs. The side or edge regions of the pile are relatively shallow in depth and relatively little or no composting activity normally occurs in these regions. Thus, the central region generates most or all of the heat created during typical composting, while the edge regions generate little or no heat.

The slab 12 is demarcated into zones or regions which correspond in part with the regions of the compost pile described above. It will be seen that in the example described herein, the size of the compost pile and the corresponding dimensions of the zones of the slab are selected as being representative of the invention and convenient in operation. These dimensions may vary considerably from those described herein. It will also be understood that unless otherwise indicated, all dimensions presented herein are approximate, since the nature of the invention permits a reasonably high level of imprecision in the handling of compost, arranging it in windrows and the like.

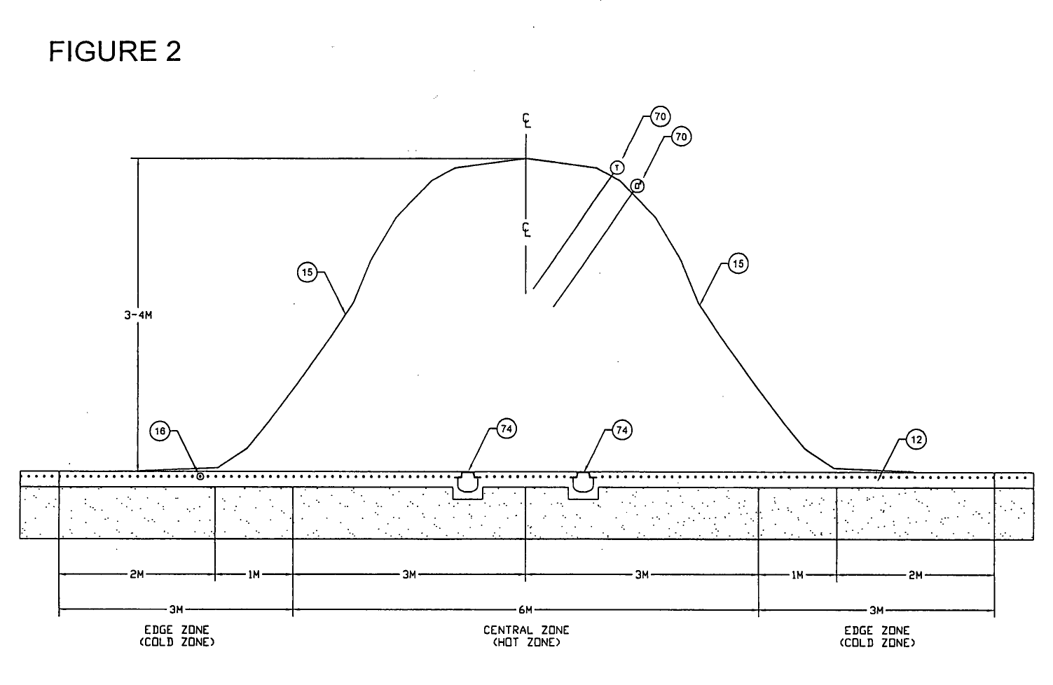

As shown in FIGS. 1a and 2, the location of the windrows on the slab 12 is selected such that each windrow is deposited upon on an 8 meter wide strip along the slab, alongside of which on either side is a further 2 or more meter wide region which represents a free space on either side of the windrow for operation of the mobile winder and other operations. The windrows 14 are deposited upon the slab such that the central zone of each pile is 6 meters wide at its base while a 1 meter wide strip on either side constitutes the edge zones. Two aeration trenches separated by 1.5 meters determine the center of each pile and extend lengthwise for substantially the length of the row region. For this application, the windrows are arranged in two groups of four parallel rows, denoted as the south and north arrays for convenience. The individual regions of the slab for receiving the respective windrows are referred to herein as rows; thus, the pad described herein has a total of 8 rows. The rows may be demarcated on the slab by the air/drainage trenches, as well as by painted lines or other ways to conveniently demarcate the location of the windrows. The rows comprise proximal and distal ends, for convenience of reference.

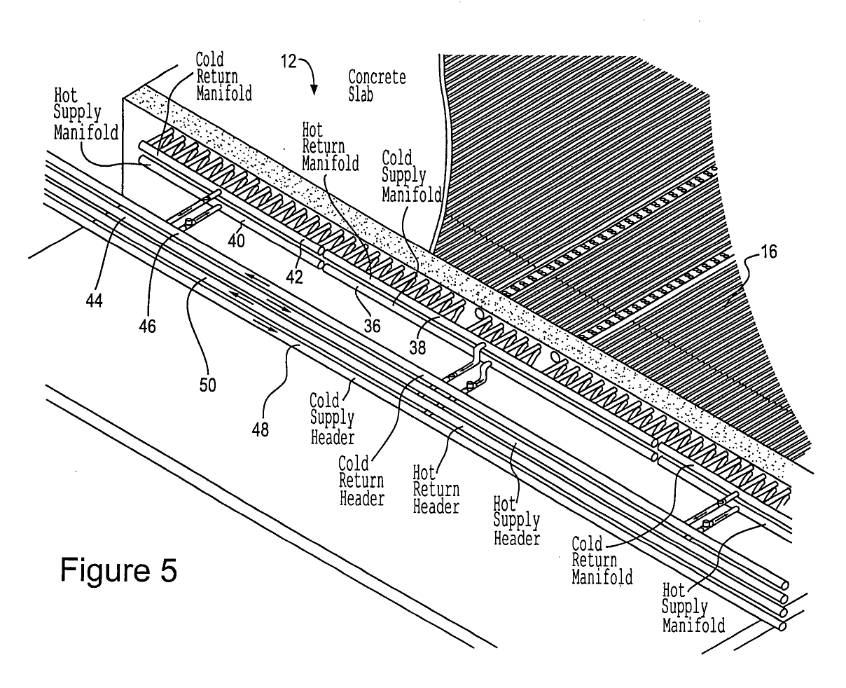

As seen in FIGS. 2 through 7, a network of pipes 16 is embedded within the slab 12 for transporting a heat transfer fluid. The fluid conveniently consists of a liquid such as a glycol/water solution. The composition of the fluid will depend in part on the expected operating temperatures; the glycol concentration will be increased for colder operating temperatures. The pipe network comprises a plurality of arrays, each associated with an individual row region of the pad, and sub-arrays as will be described below. The arrays comprise a plurality of individual pipes, for example 25 mm ID pipes, spaced parallel to each other approximately 100 mm apart within the sub-arrays. Conveniently, the pipes comprise NPS 1 polyethylene brine pipes.

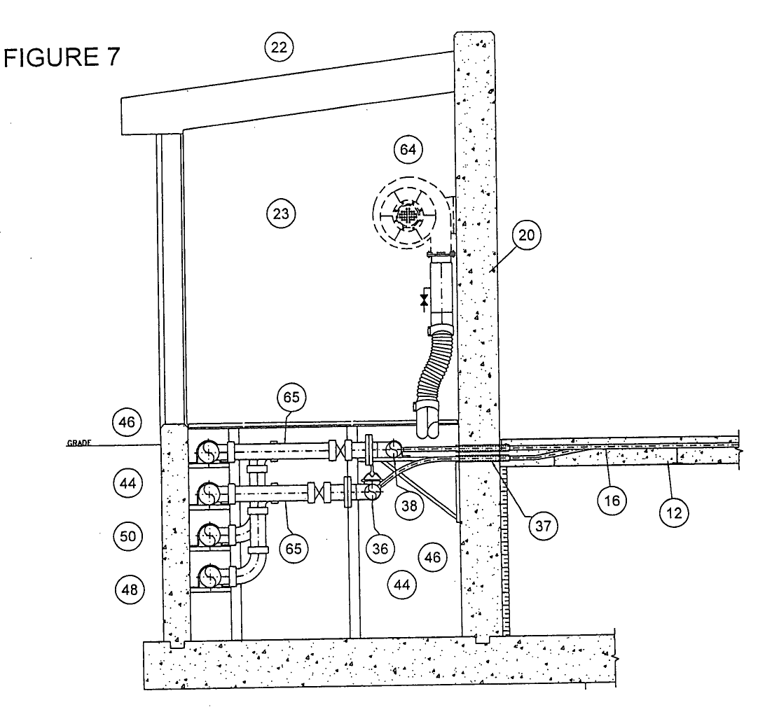

As seen in FIG. 7 a wall 20 extends upwardly along one edge of the slab, preferably adjacent to the proximal ends of the rows. The wall is provided to separate the area of biosolids handling from certain mechanical and electrical components of the system. An enclosure 22 may be provided along-side the wall on the oppose side from the slab, for housing mechanical and electrical components, as will be described in greater detail below.

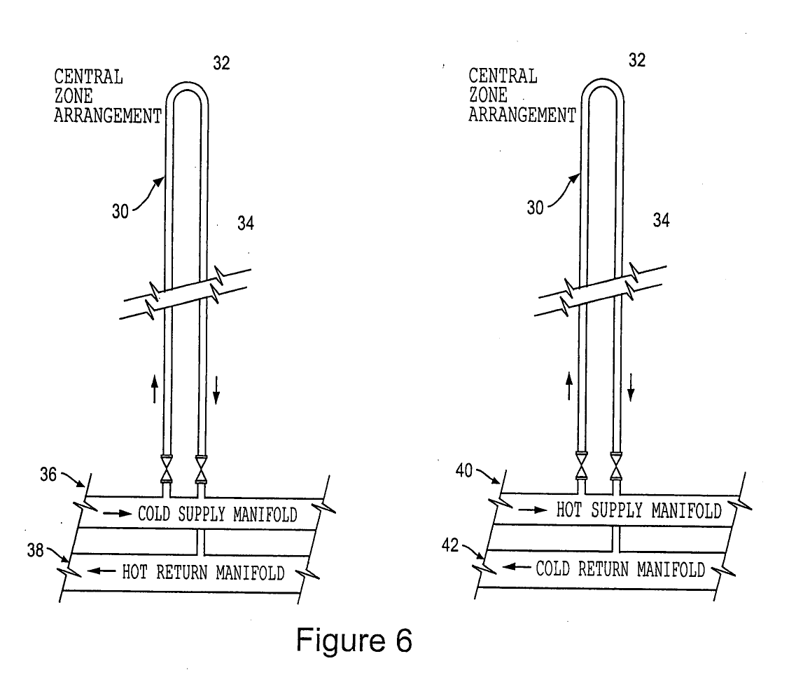

Referring to FIG. 4, the heat recovery system components are shown schematically. Each row region of the slab 12 comprises a centre (central) zone 24 and opposing edge zones 26 along either elongate side thereof. The outermost edge zones associated with the two outer rows, which are bounded by the side edges of the pad, may be somewhat wider than the edge zones which are bounded on both sides by adjacent row regions. The pipe arrays within the zones each consist of a plurality of pipes 16 arranged in parallel along the length of each zone, having aligned proximal ends and distal ends which correspond with the proximal and distal ends of the rows. The pipes in each zone are supplied with fluid by a first manifold that is associated with the zone and that extends the length of each respective zone. A second manifold receives the fluid from the pipes within each zone and comprises a return manifold. The supply and return manifolds are adjacent to each other and are all located at the proximal end of the row. The pipes 16 are arranged in a series of U's such that the openings at either end of the each pipe are adjacent to each other. As seen in FIG. 6, a first end of each pipe 16 communicates with the supply manifold and extends via a first length 30 to the distal end of the zone so as to transmit fluid from the proximal to the distal end. The pipe then attaches to a U-junction 32 at its distal end, which joins with a second length of pipe 34, so as to return fluid to the proximal end. The pipe then communicates with the return manifold to transmit the fluid to a downstream location.

By way of example, the center zone 24 is 6 meters wide and the edge zones 26 are 3 meters wide each, with the outermost edge zones at the edges of the array being somewhat wider to accommodate service vehicles at these locations. The length of the zones can vary and depends on the desired capacity of the system. A length of 50 meters is used in this particular system and corresponds to the practical limit for handling the Gore™ Cover.

The central zone 24 underlies the central, heat-generating portion of each compost windrow. This zone includes a cold supply manifold 36 for supplying cold fluid to the pipes within this zone. During the active composting phase, heat generated by the composting process is transmitted to the fluid within the centre zone, such that fluid returning to the return manifold is elevated in temperature; thus, the return manifold 38 is referred to as a hot return.

The pipes embedded within edge zones are intended to discharge heat when required so as to warm up these regions of the slab. Accordingly, the supply manifolds 40 for the edge regions each consist of a hot supply, while the return manifold 42 is a cold return, reflecting the loss of heat from the fluid into the concrete pad during operation. It will however be understood that the system is not required to operate in this fashion, but may instead vary in the order in which heat is delivered or absorbed by the respective zones.

As is shown in FIG. 7, the embedded pipes 16 extend through openings within the wall 20, so as to extend into the enclosure 22. The pipes pass through a sleeve 37 when extending through the wall 20. The supply and return manifolds, as well as the other interconnected piping and conduits described below, are contained with the enclosure 22, and are accessible to the user.

It will be seen that in an array of four windrows, there are eight hot supply manifolds 40, four cold supply manifolds 36, eight cold return manifolds 42 and four hot return manifolds 38. All of the headers are in fluid communication with corresponding trunk lines which lead to a storage tank. A first trunk line 48 carries the cold supply water, and is connected to the cold supply manifolds of the central zones of each of the sub-arrays. A second trunk line 50 carries the hot return fluid, and is connected to the hot return manifolds associated with the central zones of each sub-array. A third trunk line 44 is the hot supply pipe, and is operatively connected to the hot supply manifolds of the edge zones of each sub-array. The fourth trunk line 46 comprises a cold return pipe, and is operatively connected to all of the cold return manifolds of the edge zones of each sub-array. It will thus be seen that each of the four trunk lines is operatively connected and in fluid communication with either the central zone or the edge zones of all of the sub-arrays.

As is shown schematically in FIG. 4, the heat transfer fluid is stored in a storage tank 52. The tank 52 is a generally conventional cylindrical tank, which is preferably well insulated. The tank 52 is relatively tall in relation to its diameter so as to enhance heat stratification of the liquid within the tank, so as to permit cold liquid to be withdrawn from the tank base and hot liquid from the top portion of the tank. The cold supply pipe is thus in fluid communication with the base of the tank so as to withdraw cold fluid from this part of the tank. This pipe is pressurized by a first in-line pump 54. Shut-off valves 56 are provided within the line upstream and downstream of the pump 54. Operation of the first pump is controlled by the control system 100, which will be described in detail below. The hot return pipe 50 delivers heated liquid into the upper portion of the tank 52. The hot supply pipe 44 is in fluid communication with the uppermost portion of the tank 52, so as to draw fluid from the uppermost, and correspondingly hottest, portion of the fluid within the tank. A second pump 58 in line with the hot supply pipe 44 pressurizes the fluid within this pipe. Operation of the second pump 58 is also controlled by the control system 100, and shut-off valves 62 are provided upstream and downstream of the second pump. The cold return pipe 46 delivers the cold return liquid to a lower portion of the storage tank 52. Normally, sufficient stratification occurs within the tank without the need for any physical barriers or other means to reduce circulation.

Conveniently, the manifolds and trunk lines are housed within an enclosure 22 physically separate from the compost piles, for example within a fully enclosed optionally heated enclosure 22, as seen in FIG. 7. The enclosure 22 also house the compost pile aeration supply fans 64, for artificially aerating the compost piles. Conveniently, the concrete pad is provided at grade, with the enclosure housing the headers and pipes being below grade.

The individual supply and return manifolds are all in fluid connection with the corresponding supply and return trunk lines via connector pipes 65, as seen in FIG. 4. Each connector pipe includes a control valve 68 which in turn is operatively connected to the control system 100, for controlling operation of the valves 68 so as to individually control fluid flow within the centre and edge zones of each row.

An array of temperature sensors 66 is provided for monitoring of the heat transfer fluid temperatures at various positions within the system. The sensors 66 are all operatively connected to the central controller 100, and supply an electronic signal for indicating the detected temperature. The signals are transmitted (by wire or wireless methods) back to the control system. Temperature sensors 66 are provided within each of the connector pipes connecting the manifolds to the respective supply and return trunk lines. As well, temperature sensors are provided within the fluid storage tank 52, preferably at multiple levels within the tank to detect temperature within different portions of the tank, for example, lower, middle and upper portions. The temperatures detected by the sensors 66 may be displayed in the screen displays of the controller 100, by way of boxes displayed on the screen at a location within or adjacent to the display of the respective component or position associated with the sensors.

Sensors are also provided for detecting conditions within the compost windrows. For this purpose, each cover 15 includes two small openings. The openings permit the insertion of sensors 70 into the compost piles, in particular temperature and oxygen sensors. Sensors 70 comprise elongate rods inserted deep into the piles, to detect conditions within the pile interior. Signals from the sensors 70 are transmitted to their respective windrow controller.

Flow meters are incorporated within the pumps 54 to detect the fluid flow rates within the system. Signals from the flow meters are transmitted to the central controller so as to display the flow data to the user. Fluid pressure sensors are also provided at several locations so as to detect fluid pressure within the conduits, preferably within the region of the pumps.

A pair of aeration channels 74 (seen in FIG. 2) are recessed into the slab at each row to provide aeration to the compost windrows. The channels are encased in the concrete slab. The cover is made of cast iron and perforated with holes for supplying air and draining excess leacheate. A number of holes are plugged so as to provide an even pressure for the total length of the trench. A water trap is used at the downstream end to provide a seal and prevent air from escaping, thereby making it possible to pressurize the air trench. The channels 74 are supplied with air through conduits that are fed by the aeration fan 64. The capacity of the fan 64 is selected according to the size of the overall system, the makeup of the compost and other parameters known to the art. Aeration of the compost windrows from below enhances the aerobic bacterial activity within the piles, thereby improving the decomposition and achieving higher temperatures within the piles. The system as described herein preferably employs a positive aeration mode. A negative aeration mode may be used with the appropriate adaptations, such that air is drawn downwardly through the piles.

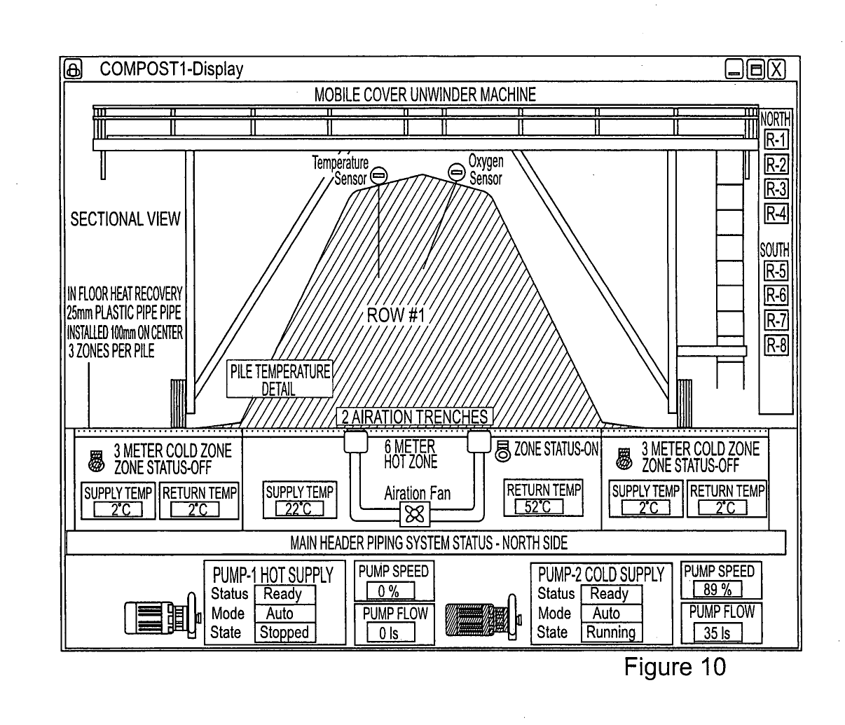

As seen schematically in FIG. 10, handling of the tarp is carried out with a mobile cover unwinding/winding unit 108. The unit comprises a movable frame which straddles the complete windrow. The frame has a sufficient height to clear the top of the windrows. The frame is supported on wheels 112, permitting it to roll along the length of the windrow. The system as a whole may be provided with one or more mobile units 108 for maximum operational flexibility. The unit 108 is highly maneuverable so as to enable it to be shifted between windrows as required. The wheel spacing permits the wheels of the unit to roll along the edge zones, and the overall width of the frame generally corresponds to the overall width of the rows and their associated cover tarps, such that the outer edges of the frame are generally aligned with the outer edges of the edge zones. The frame supports a cover winder/unwinder 114, consisting of a rotatable drum, which is hydraulically driven. The unit rolls along the windrow as it rolls or unrolls the cover. Power of the unit 108 is provided by a diesel engine which drives a hydraulic pump which then operates all systems including propulsion, steering, winding/unwinding.

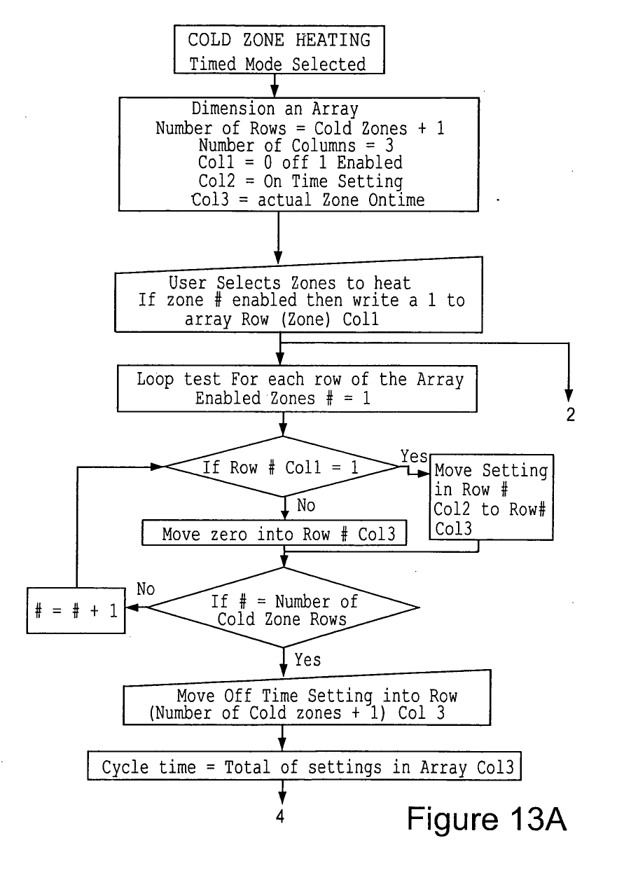

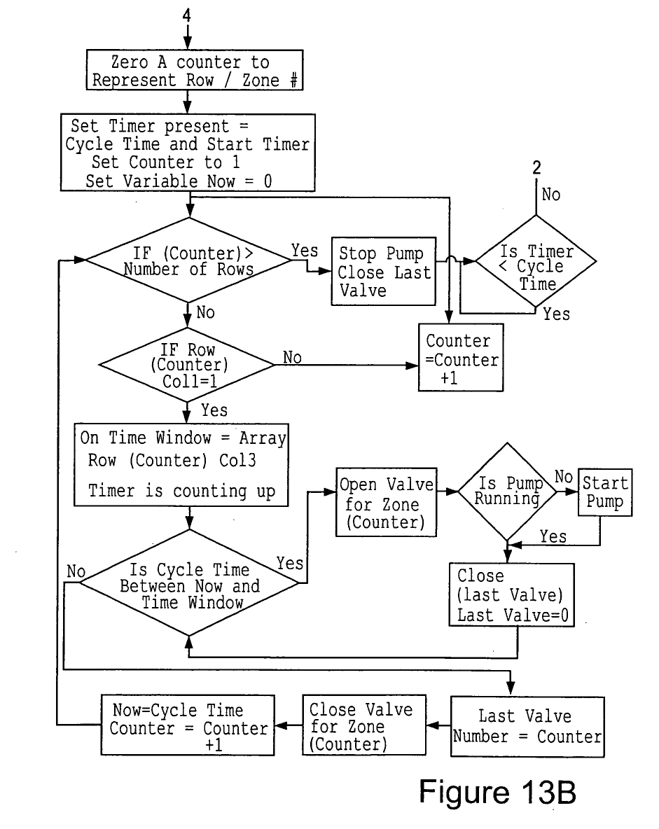

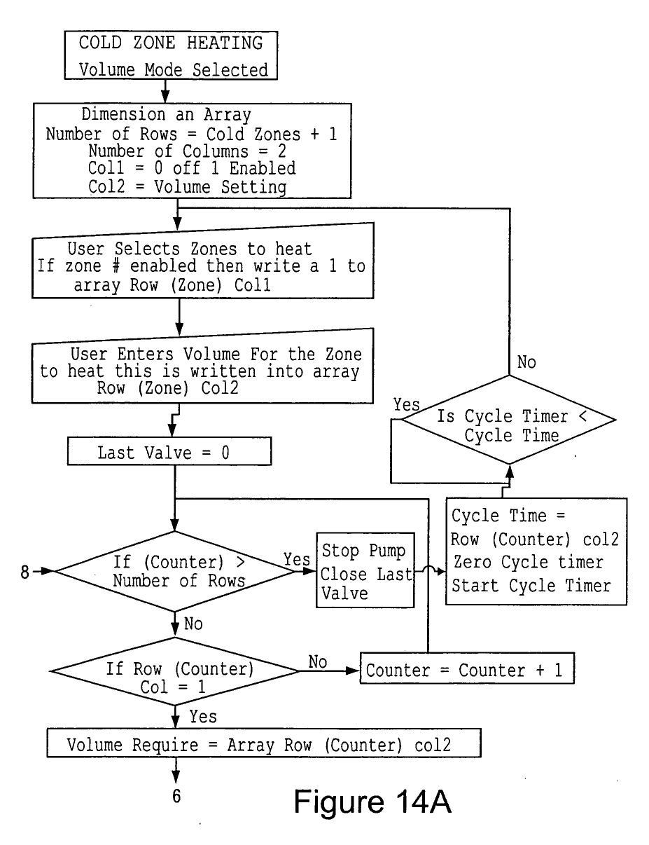

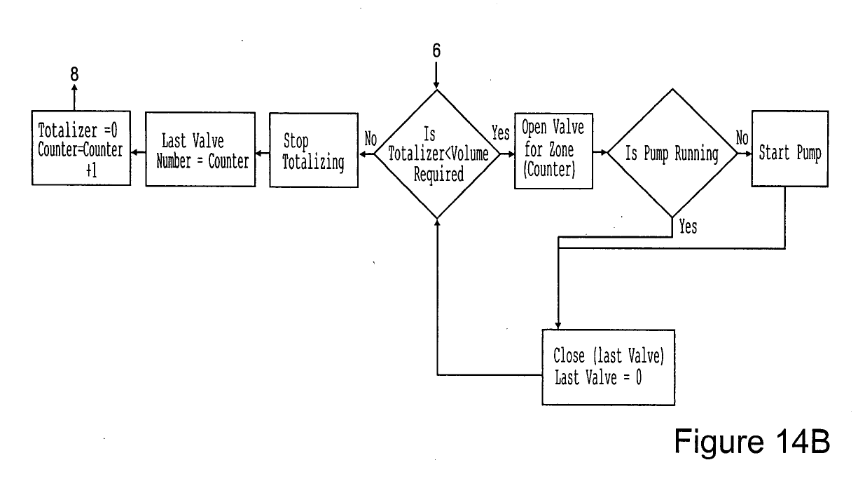

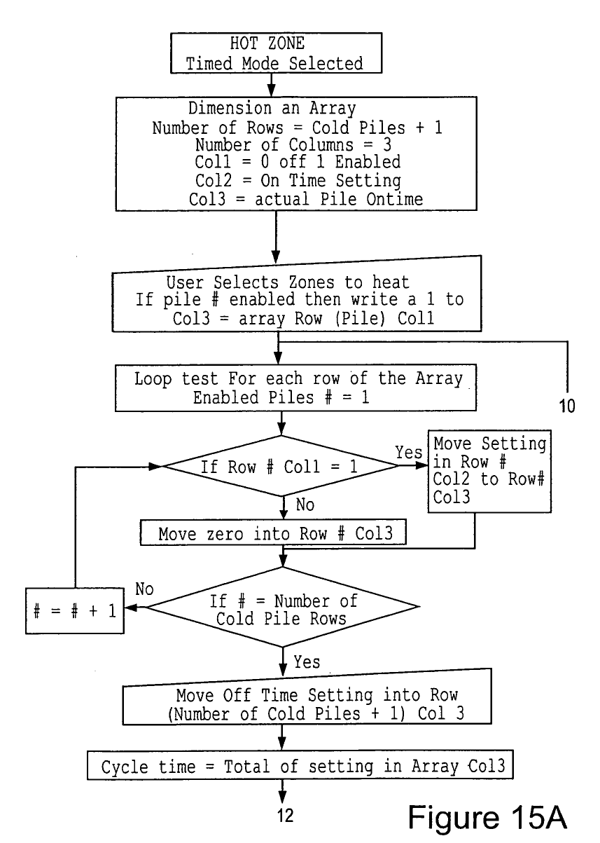

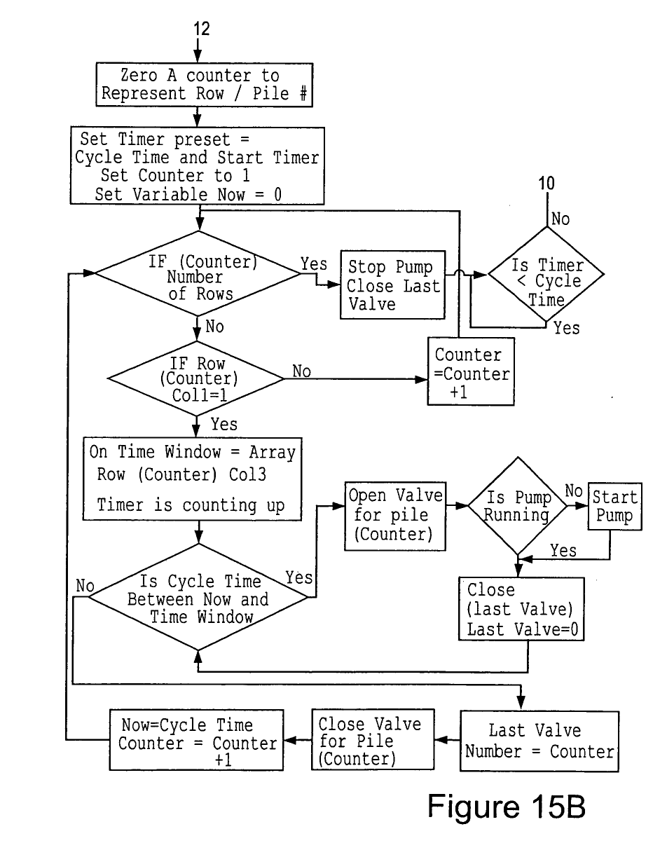

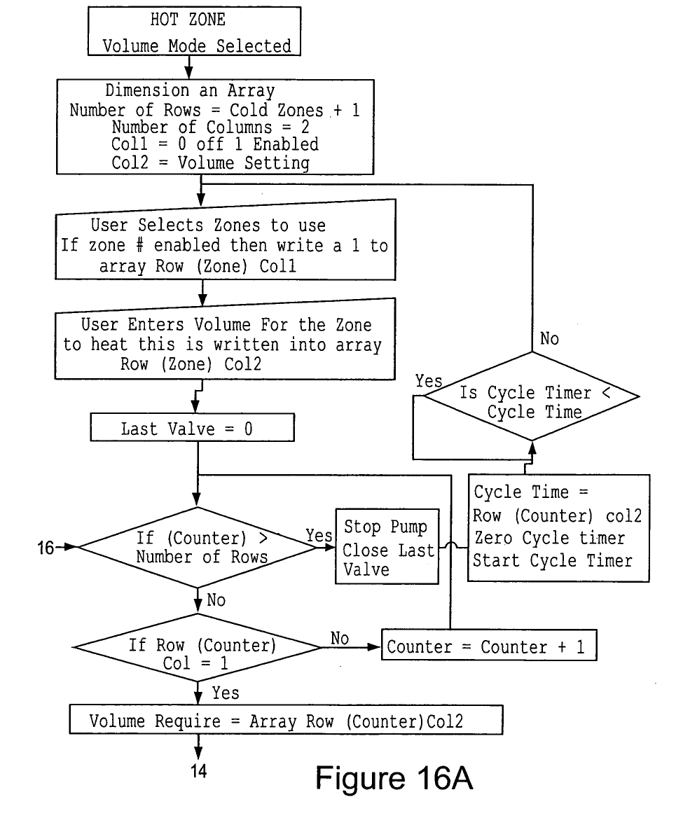

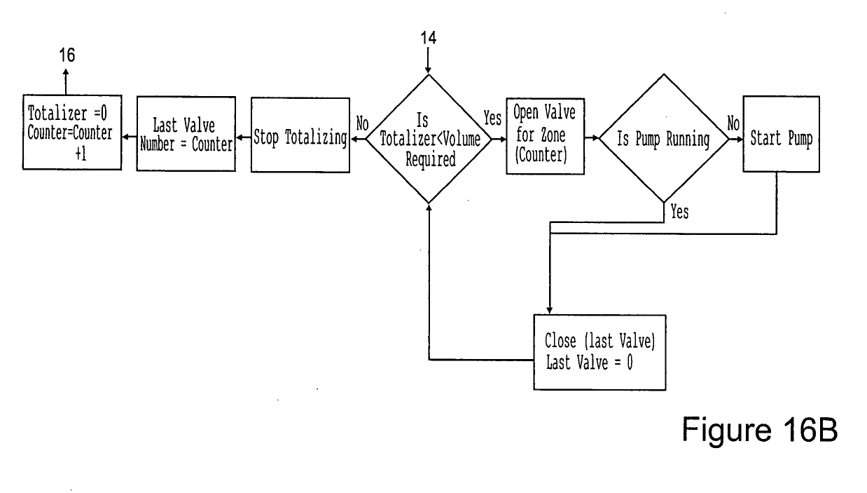

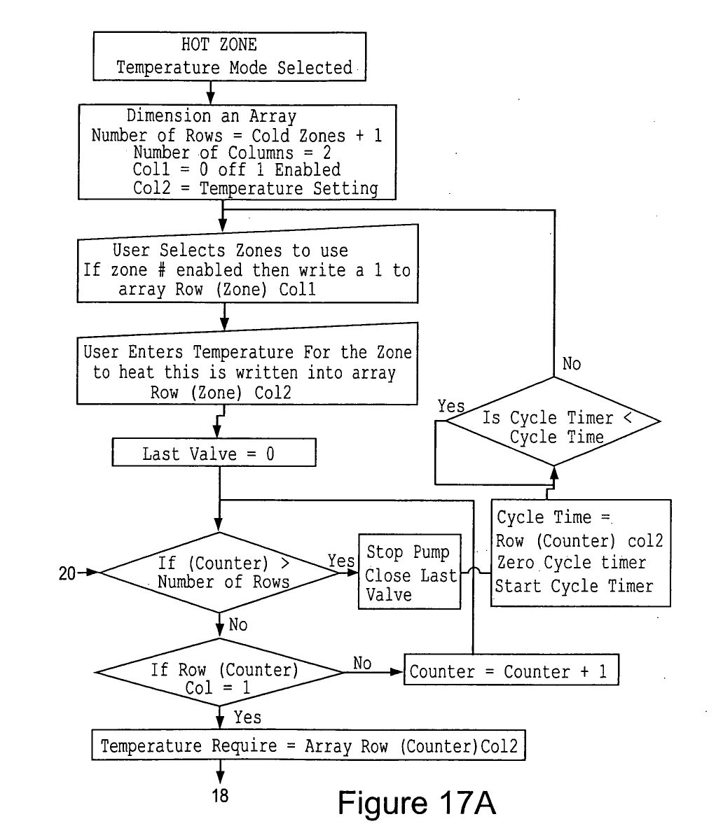

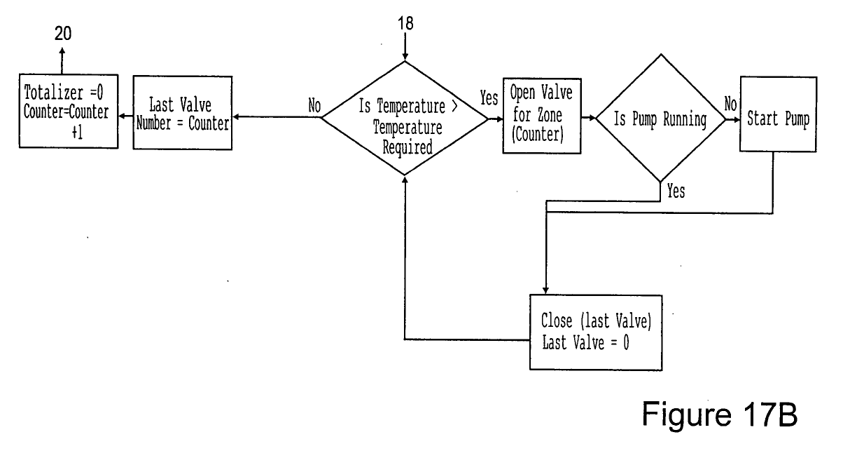

Operation of the system is controlled by the controller 100, which preferably comprises a personal computer programmed to carry out the control operations described herein. Essentially any convenient form of signal processing means may be adapted for the invention, and it will be understood that the rapidly evolving arena of computer technology may in the future permit the use of computing or signal processing systems to be employed which are currently not contemplated for this purpose. The controller includes a display screen. FIGS. 13 to 17 illustrate operation of the controller schematically by way of block diagram flow charts. The controller 100 is operatively connected to the controllable elements of the system, namely the pumps and valves described above, as well as the sensors described herein including the various temperature, pressure, flow, and oxygen sensors. The operative connections comprise any convenient means known to the art, whereby a control signal from the controller actuates the controllable elements, and optionally receives feedback from the controllable elements, and also for transmitting signals from the various sensors. It will be seen that signal transmission may occur, for example, by wired or wireless communications, and may consist of essentially any convenient process or system for transmitting data over relatively short distances. It is also contemplated that the control system may be remotely located, and that signals are transmitted over a relatively large distance.

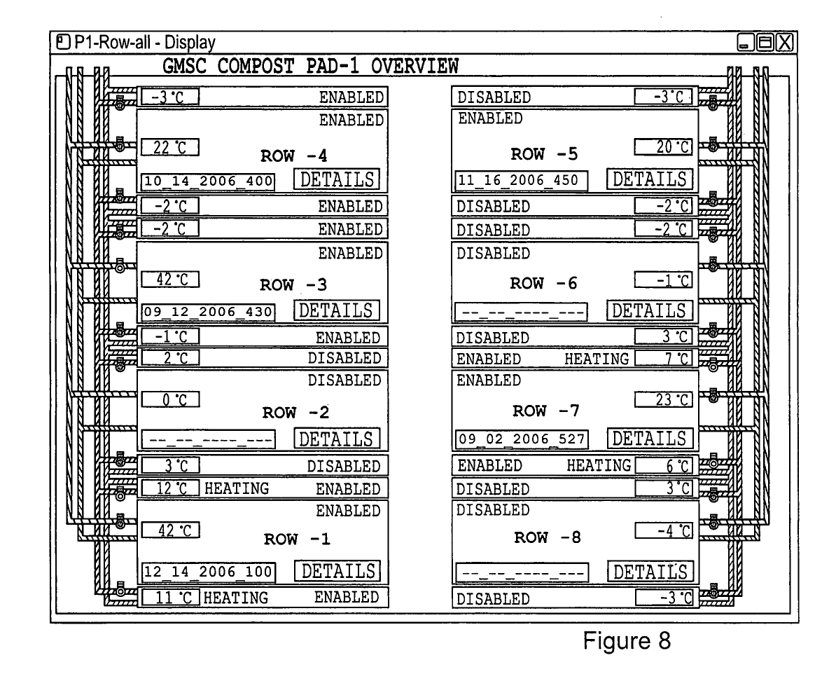

Operation of the control system is described in part herein by reference to depictions of the controller display screens, seen in FIGS. 8 through 12, which are displayed during operation of the system. The present description is by way of illustration, and it will be seen that other arrangements may be provided for the control system. Hence, the “control means” described herein are intended to encompass any suitable control system which performs high level functions similar to those described herein. The user interface of the controller may comprise a touch screen arrangement, or alternatively a conventional keyboard and mouse arrangement, or any other suitable user interface. Turning to FIG. 8, the primary operator interface display shows eight compost pile rows 14 displayed as rectangles identified as rows (1) through (8). Each rectangle is divided into three zones, the center zone 110 at the core of the windrow-shaped pile and two edge zones 112 which border the central zone along either side thereof. During the active phase of the composting process, the central zone is heat-generating.

The status of the center and edge zones is indicated on the screens. The center zone 110 is either “enabled” or “disabled”, wherein an enabled pile is one which is actively producing heat and from which heat is optionally being withdrawn from the pile via the heat recovery fluid, while a disabled pile is not in active aerobic heat production. In the enabled mode, heat recovery may be carried out via circulation of the heat recovery fluid, although as will be seen below heat recovery is selectively engaged in an enabled row. The edge zones 112 are disabled when the row is not in use and enabled when the row is in use. The user has the option to direct the circulation of heated fluid within an enabled edge zone for melting of snow and ice on the overlying cover.

The screen display is colour coded, for example with the center and edge zones being coloured grey when disabled and green, blue or orange in the enabled states. For the enabled states, the color coding indicates whether the enabled zones are in an active heat transfer state wherein the heat is being recovered from an enabled centre zone (green) or not (blue). In a similar fashion, the enabled edge zones may be displayed with color coding to indicate whether an edge zone is being warmed up by the circulation of heated fluid (orange) or not (blue). The changing of colours in each zone also provides visual display of the cycling of each zone. The selection of colors is a matter of design choice and may be varied.

Operation of the system will now be described, by reference to the control system and the controller display screens. The screens illustrated and described herein may be selectively displayed by a scrolling operation or the like.

As shown in the screens, each compost pile 14 is processed and controlled independently, with its own fluid connections and valving between the manifolds and trunk lines, in order that liquid may be circulated within each row independently of the other rows. The screen displays indicate the operational status of the various components. The valves are indicated schematically, and are depicted in a first colour when closed, and a second colour when open. In a similar fashion, the pumps are displayed schematically in a first colour when off, and a second colour when on.

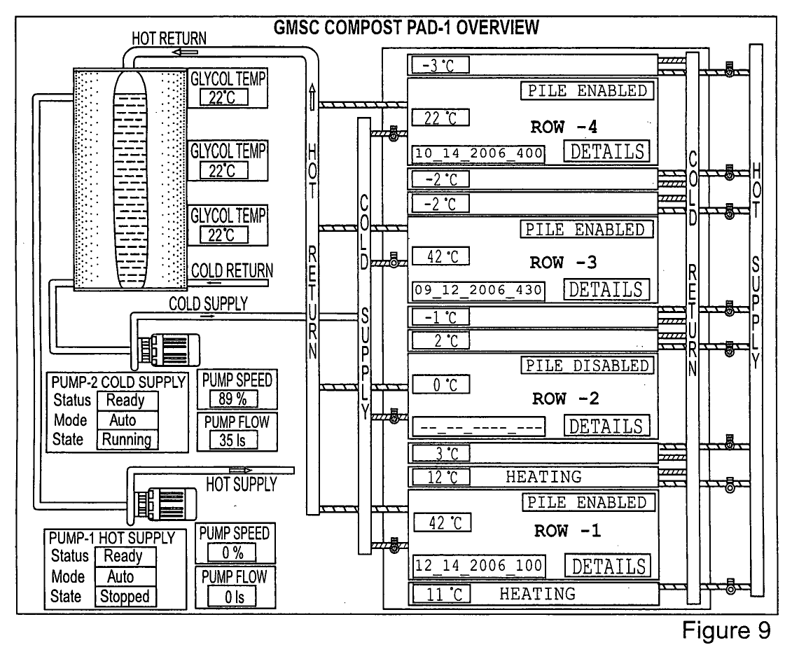

As seen in FIG. 9, fluid from the cold supply line 48 is pumped to the cold supply manifold 36, for circulation within selected regions. In the example presented herein, row (2) is disabled in that it is not actively generating heat and not in the cycling program. The lack of activity could result from the row not being in use, or if the composting within the windrow is not at the optimum operating condition. Rows (1), (3) and (4) are enabled, in that they are actively generating heat. Rows (1) and (3) indicate a temperature of 42° C., indicating active composting is occurring, while row (4) displays 22° C., indicating a less active phase of the composting. The cold supply valve 68 associated with row (3) is open, so as to direct a cold fluid supply into the centre zone of row (3). The fluid channeled beneath row (3) enters the hot return 38 associated with that row, and from there is transmitted back via the hot return trunk line 50 to the supply tank 52 for use wherever required, or storage. In the example of screen 2, hot fluid from the supply tank is being used to heat the edge zones of row (1) and for this purpose the heated fluid enters the hot supply trunk line 44, which communicates with an upper portion of the tank so as to draw from the hottest portion of the tank. The hot supply fluid may be pressurized by the second pump 58. In the example presented herein, the second pump 58 is active as heating is taking place. The hot supply fluid charges the hot supply manifold 40 of selected rows. In this example, the connector pipes to rows (2), (3) and (4) are closed. However, the valves associated with the two edge regions of row (1) are open, so as to heat the pad in the edge regions of row (1). As will be seen, the pad in this region is being heated by a glycol water solution of 11 to 12° C., so as to thaw the overlying membrane at these edge regions.

As seen in FIG. 10, the temperature and oxygen levels detected by the sensors 70 embedded within the interior of the piles are displayed. Clicking on the icon for the respective sensor will provide particulars of the temperature and oxygen levels therein or alternatively these values may be constantly displayed on the screen. FIG. 10 provides a graphic display of a single row, presented in cross-sectional view for convenience. There would be eight such screens accessible from any one screen. This view indicates the detected temperatures for each of the edge zones, in terms of supply and return temperatures of the fluids, as well as the supply and return temperatures of fluid directed through the central zone.

FIG. 10 also graphically shows the aeration fan 64, and its operation status within the selected row. FIG. 10 also depicts the mobile cover unwinder 108, which will be described below.

Individual rows 14(a)-(h) may be selected for display in screen 3, by clicking on a selected row at the upper right corner of the screen.

The lower portion of the screen depicted in FIG. 10 displays the pump status for the first and second pumps, namely the hot and cold supply trunk lines, indicating the pump speed and pump flow for each. The pumps displayed are associated to the pile also displayed. In this case, pumps (1) and (2) are used in relation to rows (1), (2), (3) and (4). The exact same arrangement is used on rows (5), (6), (7) and (8) which would be associated with pumps (3) and (4).

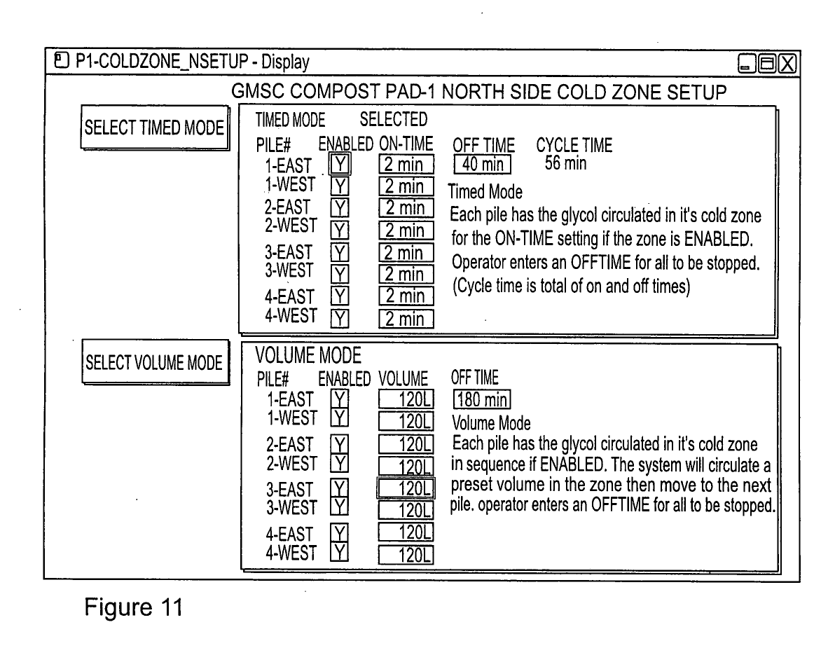

FIG. 11 depicts the user control screen for controlling the sequence of fluid circulation within the edge zones 26 of each row 14(a)-(d) which allows the operator to set-up the mode and time/volume for circulating fluid in the edge zones. The user has the option of selecting a timed mode, wherein the fluid is circulated within the edge zone for a predetermined time period for each row, or alternatively a volume mode, wherein a pre-selected volume is circulated. In order to concentrate the heating effect for melting of the overlying ice and snow on the tarp, the heated liquid may be circulated within a single edge zone at a time. In the example illustrated in FIG. 11, the entirety of the heated fluid for one side of the system is circulated within a single edge zone at a time, for a two minute interval. In the example herein, the north side of the pad includes four rows, with each row having two edge zones, for a total of eight edge zones each receiving two minutes of fluid circulation. Following fluid circulation to the eight edge zones, the user may select an off time to permit reheating of the fluid. The total cycle time in the example herein is fifty-six minutes, comprising sixteen minutes of heating (eight edge zones receiving two minutes each), followed by an off time cycle of forty minutes. In the second example shown in FIG. 11, the user may alternatively select a volume mode. In this example, each edge zone receives 120 litres of heated fluid, followed by a selected off time, which in this case is selected as 180 minutes.

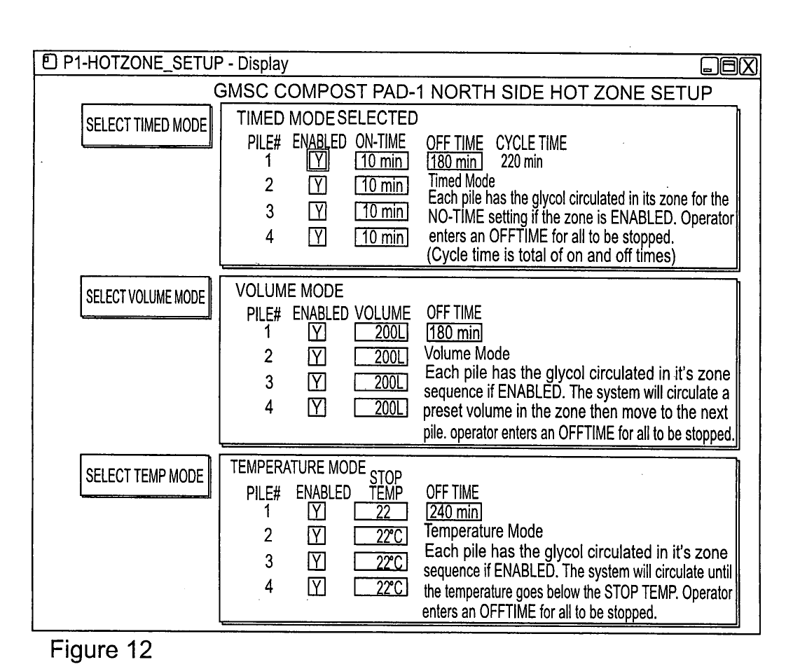

FIG. 12 depicts a graphic screen display, which enables the user to control fluid circulation within the central zones 24 for each of rows 14(a)-(d), and allows the operator to set-up heat recovery from each windrow central zone based on time, volume, or temperature. These zones are at locations where the composting biosolids are generating heat from the composting process, and these zones are thus each referred to as the “hot zone” of each row. The user has three options for controlling fluid circulation in this zone, either in a timed mode, volume mode or temperature mode. In the timed mode, fluid is circulated within each hot zone for a selected time, in this case ten minutes for each zone. Following circulation within all four zones, an off time is optionally selected to permit reheating of the pad, in this case selected as 180 minutes. The total cycle time is thus 220 minutes. In the alternative volume mode, the user selects a volume for each row, in this case 200 litres for each row, followed by 180 minutes off time. In the temperature mode, the user selects a predetermined temperature, and fluid circulates within each zone for so long as the temperature is higher than the selected stop temperature. Each row has the fluid circulated in its zone in sequence until the temperature of the glycol solution exiting the zone drops below the selected stop temperature, at which time fluid is then circulated within the second such zone, and so forth. Following circulation within all zones, the circulation stops for a selected off time.

It will be seen that within both a cold zone and hot zone setup, the user may select an identical value for all of the rows, or alternatively different times for the rows, depending upon pile size and other parameters.

The system may also be adapted to supply heated fluid to regions other than selected edge zones. For example, the heated fluid may be directed to the central zone of a selected row in order to assist commencement of aerobic bacterial action.

The control logic associated with all functions of the fluid circulation system is depicted by bloc flow diagrams included in FIGS. 13 through 17, which schematically illustrate the operation of the control system 100.

The detailed embodiments and other description of the invention described and illustrated herein are not limitative of the scope of the invention. Rather, the full scope of the invention is defined by this patent specification as a whole, including without limitation the patent claims presented herein, and further including structural and functional equivalents of any elements described and claimed in this patent specification.

1. A system for composting of biosolids, comprising:
a) a pad for supporting at least one pile of said biosolids, said pad defining at least one row region for receiving a corresponding pile on the surface thereof each row region comprising a central zone and one or more edge zones adjacent thereto,
b) a source of heat-retaining fluid suitable for transferring heat between said zones;
c) an array of pipes associated with said pad, in fluid communication with said source, said array comprising a first sub-array located within said central zone, and a second sub-array located within said edge zone, each sub-array being independently connected to said source, wherein said array and pad are configured to permit transfer of heat between said pad and said fluid circulating within said pipes;
d) at least one valve means for selectively controlling the flow of said fluid through said array;
e) at least one pump means for circulating said fluid through said pipes and said source;
f) at least one temperature sensor within each of said central zone and said edge zone for transmitting signals in real time indicative of the temperature of said pad, said fluid or said biosolids pile;
g) a controller for controlling operation of said system, said controller including means for selectively controlling the circulation of said fluid from said central zone to other zones of said pad so as to transfer heat generated by aerobic decomposition of said biosolids pile within said central zone, to a zone in need of such generated heat; and
h) signal transmission means between said controller and said temperature sensor, said pump means and said valve means.

2. The system as defined in claim 1 further comprising a removable cover comprising a flexible membrane for selectively covering said biosolids pile, said cover being dimensioned to extend over said central zone and at least a portion of said at least one edge zone for contacting the pad within said at least one edge zone.

3. The system as defined in claim 1, comprising a plurality of row regions, wherein said controller selectively controls circulation of said fluid between said row regions.

4. The system as defined in claim 1 wherein said zone in need of heat comprises at least one of said edge zones.

5. The system as defined in claim 1 comprising multiple row regions wherein said zone in need of heat comprises a central zone of a row region remote from the source of said heat.

6. The system as defined in claim 1, wherein said fluid source includes an insulated liquid storage tank for storing heated liquid for use within said system, said temperature sensors including at least one sensor associated with said tank for detecting the temperature of the liquid therein.

7. The system as defined in claim 6, wherein each said sub-array is independently joined to said tank by a conduit which communicates with said tank at a selected height wherein each sub-array associated with said edge zone receives said liquid from an upper portion of said tank and each said central zone receives liquid from a lower portion of said tank, wherein said height is selected such that vertical stratification of liquid within said tank arising from differential temperatures of said liquid therein promotes transfer of heat from said central zone to said edge zone.

8. The system as defined in claim 1, wherein said sub-arrays each include at least two manifolds, the sub-array within said central zone comprising a cold supply manifold and a hot return manifold, each said edge zone comprising a cold supply manifold and a hot return manifold, said manifolds being independently in fluid communication with said source for circulating fluid from each said manifold independently of others of said manifolds.

9. The system as defined in claim 8, comprising a plurality of said central and edge zones, wherein all of said cold supply manifolds are in fluid communication with a common cold supply trunk line, all of said hot return manifolds are in fluid communication with a common hot return trunk line, all of said hot supply manifolds are in fluid communication with a hot supply trunk line, and all of said cold return manifolds are in fluid communication with a cold return trunk line, with each of said trunk lines being in fluid communication with said fluid source.

10. The system as defined in claim 1, wherein said controller includes a visual display for displaying a graphic illustration of said at least one row region, including the central and edge zones thereof, the temperatures detected by said temperature sensors, the on/off status of the pump and valve means, and the enabled/disabled status of the central and edge zones, wherein an enabled zone comprises a heat-generating actively composting pile, and a disabled zone is indicative of an absence of an actively composting pile.

11. The system as defined in claim 10, wherein said temperature, on/off status and enablement status are displayed in real time, and said controller permits user control over said system in response to said real time information.

12. The system as defined in claim 10, further comprising a data logger.

13. The system as defined in claim 10, further comprising a user control for selecting display information relating either said system in its entirety, a portion of said system or an individual compost pile region.

14. The system as defined in claim 10, wherein said control system selectively controls operation of said system according to either of a timed mode or a fluid volume mode, wherein fluid is circulated within each selected zone for a selected time period, or a selected volume of fluid is circulated.

15. The system as defined in claim 14, comprising multiple edge zones and wherein said selected mode includes selection of serial and sequential application of heat, wherein heat is sequentially and serially applied to selected edge zones, followed by a selected recovery time.

16. The system as defined in claim 15, wherein said controller selectively controls circulation of said fluid within said central zone in one of a selected timed mode, volume mode or temperature mode, wherein said timed mode permits circulation of said fluid within said central zone for a selected time, said volume mode permits circulation of a selected volume of fluid within each of said central zones, and said temperature mode permits circulation of fluid within said central zone for seas long as the detected temperature exceeds a selected minimum.

17. The system as defined in claim 16, comprising a plurality of said regions, wherein said selected mode selectively circulates said fluid according to said selected mode serially within each selected region in turn, optionally followed by a selected recovery period.

18. The method for composting of biosolids, comprising the steps of supplying a system comprising:
a) a pad for supporting at least one pile of said biosolids, said pad defining at least one row region on the surface thereof for receiving a corresponding pile, each row region comprising a central zone and one or more edge zones adjacent thereto;
b) a source of heat-retaining fluid suitable for transferring heat between said zones;
c) an array of pipes associated with said pad, in fluid communication with said source, said array comprising a first sub-array located within said central zone, and a second sub-array located within said edge zone, each sub-array being independently connected to said source, wherein said array and pad are configured to permit transfer of heat between said pad and said fluid circulating within said pipes;
d) at least one valve means for selectively controlling the flow of said fluid through said array of pipes;
e) at least one pump means for circulating said fluid through said pipes and said source;
f) at least one temperature sensor within each of said central zone and said edge zone for transmitting signals in real time indicative of the temperature of said pad, said fluid or said biosolids pile;
g) supporting at least one pile of said biosolids on said central region of said pad; and
h) controlling circulation of said fluid by receiving signal information from said temperature sensors and selectively controlling the circulation of said fluid in response to said signal information wherein said fluid is selectively transferred from said at least one central zone, to a zone of said pad different from said central zone so as to transfer heat generated by aerobic decomposition of said compost within said central zone, to another zone of said pad in need of such generated heat.

19. The method as defined in claim 18, further comprising positioning a flexible cover to substantially overlie said pile and at least a portion of said edge zone, said step of transferring heat comprising transferring heat from said central zone to said edge zone covered by said cover, and thereby increasing the temperature of said cover where it overlies said edge zone.

20. The method as defined in claim 18, further comprising providing a plurality of said row regions, and selectively transferring heat from one of said row regions to another of said row regions in need thereof.

21. The method as defined in claim 18, wherein said fluid source comprises an insulated liquid storage tank, each said sub-array being independently joined to said tank by a conduit in fluid communication with said tank at a selected height wherein said height is selected such that vertical stratification of liquid within said tank provides delivery of warmer liquid to said edge zone and cooler liquid to said central zone.

22. The method as defined in claim 18, wherein said step of controlling said fluid circulation includes providing a visual display and graphically displaying thereon said at least one row region, including the central and edge zones thereof, the temperatures of said zones, the on/off status of the pump and valve means, and the enabled/disabled status of the central and edge zones, wherein an enabled zone comprises an actively composting pile, and a disabled zone is indicative of an absence of an actively composting pile.

23. The method as defined in claim 22, wherein said temperature, on/off status and enablement status is displayed in real time, and said step of controlling fluid circulation permits user control over said system in response to said real time information.

24. The method as defined in claim 22, including providing user control for selecting display information relating to one or more of said system in its entirety, a portion of said system or an individual compost pile.

25. The method as defined in claim 18, wherein said control system selectively controls operation of said system according to either of a timed mode, wherein fluid is circulated within a selected zone for a selected time period, or a volume mode wherein a selected volume of fluid is circulated within a selected zone.

26. The method as defined in claim 25, comprising multiple edge zones, wherein said selected mode includes selection of serial and sequential application of heat, wherein heat is serially and sequentially applied to selected edge zones, followed by a selected recovery time.

27. The method as defined in claim 26 wherein said controller selectively controls circulation of said fluid within said central zone in one of a selected timed mode wherein said fluid is circulated within a selected central zone in accordance with a selected mode, said mode comprising one of a time mode, a volume mode wherein a selected volume of fluid is circulated within said selected central zone, or a temperature mode wherein said fluid is circulated within said selected central zone for seas long as the detected temperature exceeds a selected minimum.

28. The method as defined in claim 18 comprising a plurality of said row regions and said fluid is circulated serially and sequentially within each selected region, optionally followed by a selected recovery period.
